(19) United States
(12) Patent Application Publication
(10) Pub. No.: US 2010/0087716 A1
(43) Pub. Date: Apr. 8, 2010

(54) Portable microchip module for instantaneous analysis of bodily fluids

(76) Inventor: Ramses S. Nashed, Tierra Verde, FL (US)

Correspondence Address: Ramses S. Nashed, 505 First Avenue South, Tierra Verde,, Tierra Verde, FL, 33715, US

(21) Appl. No.: 12/287,321

(22) Filed: Oct. 8, 2008

(51) Int. Cl. A61B 5/145

(52) U.S. Cl. 600/309

(57) ABSTRACT

A portable microchip substrate supported in a receptacle having one end connected to a catheter inserted in the body of a patient. The other end of the receptacle is connected to a needleless syringe for drawing a bodily fluid sample (e.g. blood) through the receptacle and into contact with the substrate. The substrate is adapted to instantaneously perform diagnostic tests on the fluid sample in contact therewith within the receptacle. A communication device on the receptacle transmits signals to an analyzer which includes a display for indicating various diagnostic characteristics of the bodily fluid sample in real time. The receptacle and the substrate may be cleaned (sterilized) for re-use or may be disposed of after a one-time use. In an alternatively preferred embodiment, the substrate and/or communication device are directly carried on and supported by the insertable catheter.

### BACKGROUND OF THE INVENTION

1. Field of the Invention

The present invention relates generally to devices for analyzing the physical and chemical characteristics of bodily fluids, such as blood, for example, on situ and spontaneously, and, more particularly, to such devices that are adapted to be inserted into the bloodstream of a patient.

2. Description of the Prior Art

Substrates for analyzing a bodily fluid such as blood are well known in the art. For example, in U.S. Pat. No. 5,014,718, there is described a “test strip 19” provided with reagents for analyzing a blood sample. Similarly, in U.S. Pat. No. 6,544,475, there is described the use of various sensors and a microprocessor for analyzing the results of a reaction between a sample (e.g. blood) on a test strip with reagents on the test strip.

During surgery, and in other medical scenarios, it is important to be able to instantaneously analyze a wide variety of physical and chemical characteristics of bodily fluids, such as blood for example, to obtain an immediate indication of the patient's condition. Prior practice is take a blood sample and send it to the lab for analysis. This often takes hours to accomplish and deprives the surgical team of having the required information when the patient is “on the operating table” so to speak. Such information may comprise blood chemistry, enzyme levels, cell counts, medication concentrations, and so on. A need exists therefore for a device that may be inserted into the patient's body to sample bodily fluid and substantially instantaneously analyze the physical and chemical characteristics of the sample in order to provide a substantially instantaneous readout of sought-after required information. That need is met by the present invention.

### SUMMARY OF THE INVENTION

To achieve the foregoing and other advantages, the present invention, briefly described, comprises a portable microchip substrate supported in a receptacle having one end connected to a catheter inserted in the body of a patient. The other end of the receptacle is connected to a needleless syringe for drawing a bodily fluid sample (e.g. blood) through the receptacle and into contact with the substrate. The substrate is adapted to instantaneously perform diagnostic tests on the fluid sample in contact therewith within the receptacle. A communication device on the receptacle transmits signals to an analyzer which includes a display for indicating various diagnostic characteristics of the bodily fluid sample in real time. The receptacle and the substrate may be cleaned (sterilized) for re-use or may be disposed of after a one-time use. In an alternatively preferred embodiment, the substrate and/or communication device are directly carried on and supported by the insertable catheter.

The above brief description sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto.

In this respect, before explaining a number of preferred embodiments of the invention in detail, it is understood that the invention is not limited in its application to the details of the construction and to the arrangements of the components set forth in the following description or illustrated in the drawings. The invention is capable of other embodiments and of being practiced and carried out in various ways. Also, it is to be understood, that the phraseology and terminology employed herein are for the purpose of description and should not be regarded as limiting.

As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

It is therefore an object of the present invention to provide a new and improved portable microchip module for instantaneously analyzing bodily fluids which has all of the advantages of the prior art and none of the disadvantages.

It is another object of the present invention to provide a new and improved portable microchip module for instantaneously analyzing bodily fluids which may be easily and efficiently manufactured and marketed.

It is a further object of the present invention to provide a new and improved portable microchip module for instantaneously analyzing bodily fluids which is of durable and reliable construction.

An even further object of the present invention is to provide a new and improved portable microchip module for instantaneously analyzing bodily fluids which is susceptible of a low cost of manufacture with regard to both materials and labor, and which accordingly is then susceptible of low prices of sale to the consuming public, thereby making such camouflage article and method available to the buying public.

Still yet a further object of the present invention is to provide a new and improved portable microchip module for instantaneously analyzing bodily fluids which module is supported on a syringe adapted to be connected to a venous catheter.

Yet still another object of the present invention is to provide a new and improved portable microchip module for instantaneously analyzing bodily fluids which module is supported on the tip of a venous catheter.

Still another object of the present invention is to provide a new and improved portable microchip module for instantaneously analyzing bodily fluids and which communicates such information to a computer for regulating the introduction of medications or the like into a patient's body in response to the analyzed information.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and the above objects as well as objects other than those set forth above will become more apparent after a study of the following detailed description thereof. Such description makes reference to the annexed drawing wherein:

FIG. 1 is a schematic diagrammatic view of an IV system showing the portable microchip substrate of the invention supported in a receptacle which in turn is connected to a valve port in IV tube or conduit.

FIG. 2 is an enlarged view of the substrate receptacle shown in the circle marked by the number 2 in FIG. 1.

FIG. 3 is a cross-sectional view taken along line 3-3 of FIG. 2.

FIG. 4 is an elevational view of an alternative embodiment of microchip support receptacle having a wireless signal transmission device mounted thereon.

FIG. 5 is elevational view of a wireless analyzer adapted to be used with the alternatively preferred microchip support receptacle of FIG. 4.

FIG. 6 is schematic diagram showing various venous catheters used on the human body.

FIG. 7 is an enlarged schematic view of an alternatively preferred embodiment of the present invention wherein the tip of a venous catheter is inserted into a vein and wherein the catheter tip supports a substrate and communication device according to the present invention.

FIG. 8 is a cross-sectional view in elevation taken along line 8-8 of FIG. 7 and showing the substrate and communication device of the invention carried interiorly of the catheter lumen.

FIG. 9 is a cross-sectional view in elevation taken along line 8-8 of FIG. 7 and showing the substrate and communication device of the invention carried exteriorly of the catheter lumen.

### DESCRIPTION OF THE PREFERRED EMBODIMENT

With reference to the drawings, a new and improved portable microchip module for instantaneously analyzing a bodily fluid and embodying the principles and concepts of the present invention will now be described.

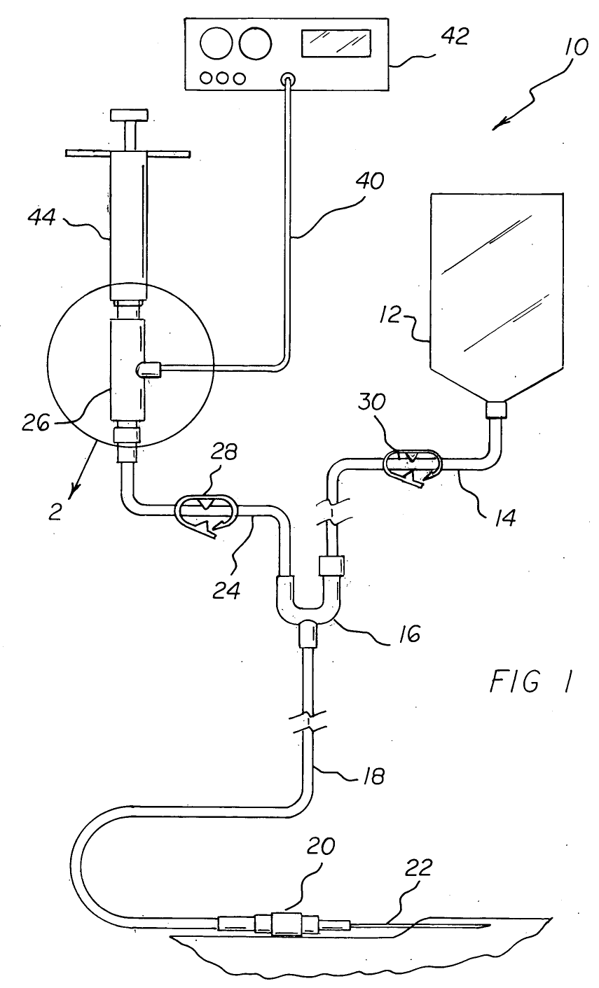

Turning initially to FIGS. 1-3, there is shown a preferred embodiment of the portable microchip module apparatus of the invention disposed in an IV catheter set-up generally designated by reference numeral 10. In each of the Figures, reference numerals are shown that correspond to like reference numerals that designate like elements shown in other Figures.

In the preferred embodiment, set-up 10 comprises an IV bag 12 connected through a flexible fluid conduit or tube 14 to one branch of a y-connector 16. The output of the y-connector 16, in turn, is connected through a second flexible tube or fluid conduit 18. Distally connected to the second tube 18 via a suitable connector 20 is a conventional angio-catheter 22 which is shown schematically in FIG. 1 embedded in the body of a patient preferably in the vein of a patient, such that fluids may be passed into the patient's bloodstream as is well known in the medical art. The other branch of y-connector 16 is connected to a third flexible tube or conduit 24 the distal end of which is removably connected to the bottom of a receptacle 26. Flow of fluids through the circuit and more specifically through tubes 14, 18 and 24 may be controlled by activation of conventional clamps 28, 30 as will be made more apparent below.

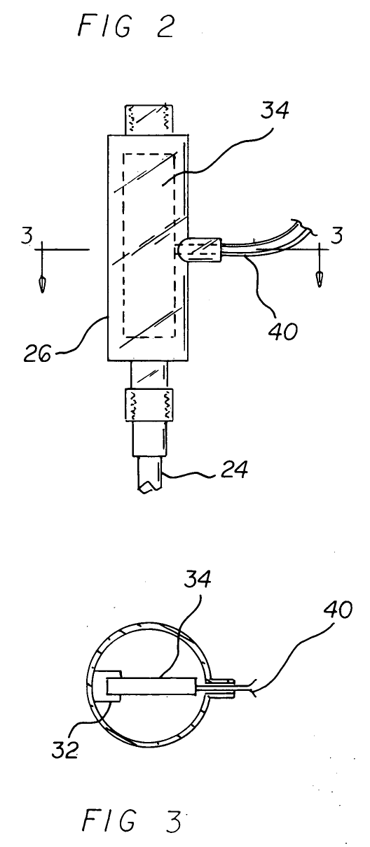

In accordance with the present invention, and as best seen in FIGS. 2-3, there is supported inside receptacle 26 by means of a suitable bracket assembly 32, a microchip substrate 34 of the type adapted to analyze the characteristics of a fluid coming into contact with the substrate, such as human blood, for example. Together receptacle 26, bracket assembly 32 and microchip substrate 34, form a unitary component or module. The top portion of the module also preferably includes a valveport (not shown) for receiving in suitable mating 10 engagement therewith the nose of a needleless syringe 44 (FIG. 1) the purpose of which will be more fully explained below. Such valveports and needleless syringes are well known in the medical arts and the details of same form no part of the present invention.

Substrates for analyzing a bodily fluid such as blood are well known in the art. For example, in U.S. Pat. No. 5,014,718, there is described a “test strip 19” provided with reagents for analyzing a blood sample. Similarly, in U.S. Pat. No. 6,544,475, there is described the use of various sensors and a microprocessor for analyzing the results of a reaction between a sample (e.g. blood) on a test strip with reagents on the test strip. The disclosure of each of the foregoing patents is hereby incorporated herein and made part of this specification by this reference.

In accordance with the present invention the microchip substrate 34 of the present invention comprises a collection of suitable sensors and microprocessors for analyzing the characteristics of the blood or other fluid sample coming into contact therewith inside receptacle 26. Upon such contact, the substrate 34 is adapted to generate suitable electrical signals conforming to the various detected blood or other fluid characteristics which are then transmitted along a suitable conductor or conductors indicated by reference sign 40 (FIGS. 1-3) to an analyzer 42. The latter is adapted suitably to convert the transmitted signals into intelligible indications of the detected characteristics of the sample being analyzed which are then displayed by the analyzer 42 substantially simultaneously in real time.

To use the apparatus of FIG. 1-3, when it is desired to analyze the bodily fluid of a patient undergoing surgery, for example, and to obtain an immediate real time analysis of such fluid, it is merely necessary to couple a conventional needleless syringe 44 to the valveport on the top of receptacle 26 and to withdraw the plunger thereof. This is done only after closing clamp 30 and opening clamp 28. Withdrawal of the syringe's plunger causes a sample of say blood, for example, to pass through the angio-catheter 22, second tube 18, the second branch of the y-coupler 16, the third tube 24, and into the interior space defined by receptacle 26 where the fluid will surround and come into contact with the microchip substrate 34 mounted therein (FIG. 3). The microchip substrate substantially immediately sends sensed information via conductors 40 to analyzer 42 where the desired characteristics may be displayed, read and recorded all in real time.

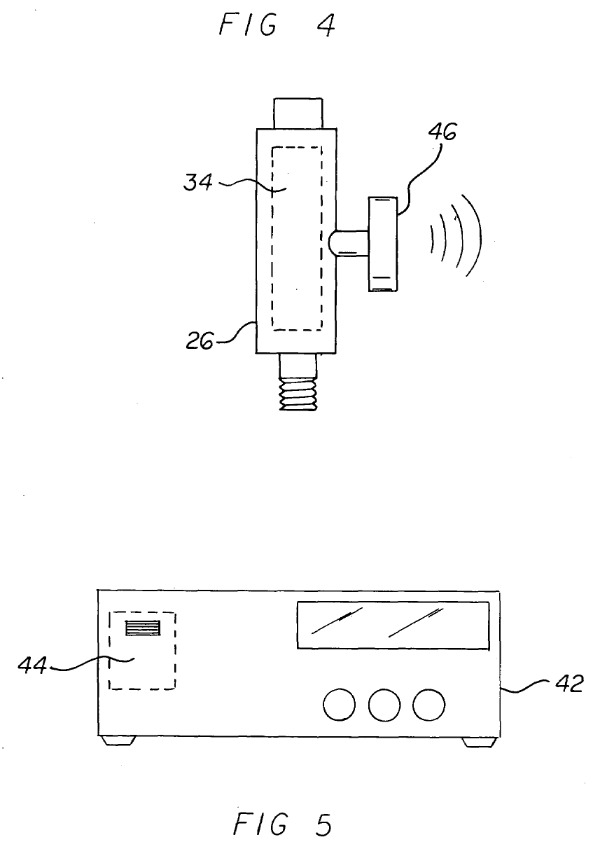

It will be appreciated that it is not necessary to use a hard-wire connection between the microchip substrate or receptacle and the analyzer. Instead, as indicated in FIGS. 4 and 5, a wireless transmitter 46 may be mounted on the receptacle (FIG. 4) and a receiver 44 may be provided in analyzer 42 thereby dispensing with conductors 40.

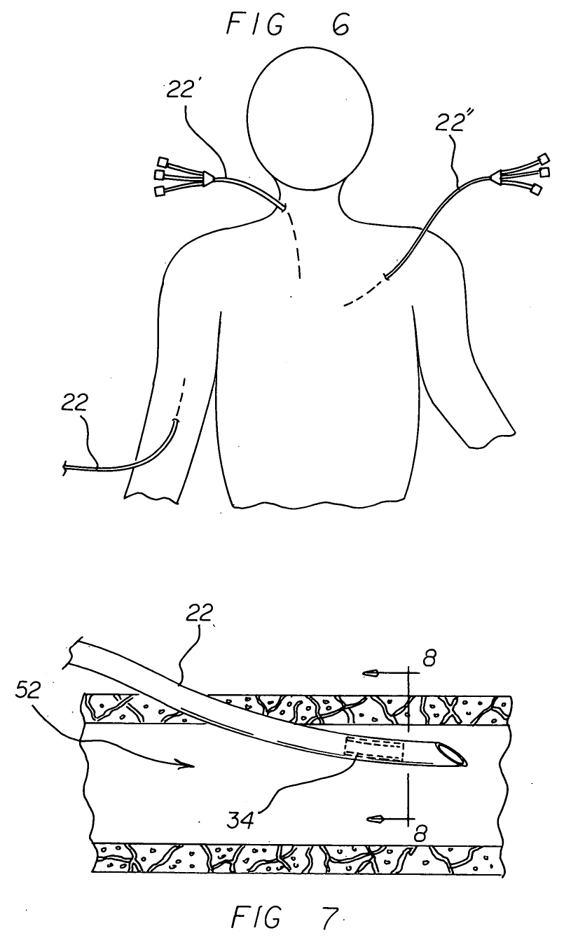

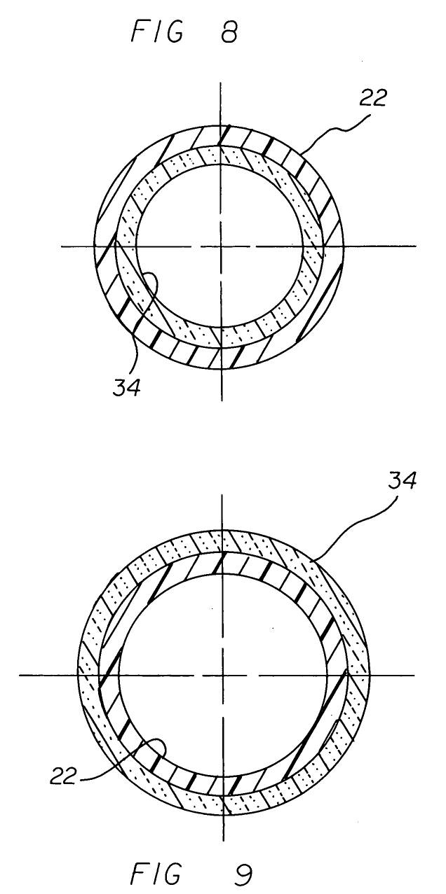

In an alternatively preferred embodiment of the invention, substrate 34 may be carried directly on the tip of the catheter 22. This alternate arrangement is depicted in FIGS. 7 through 9 where it will be observed substrate 34 is depicted in the form of a tubular member suitably affixed on the inside or interior wall of the lumen of catheter 22 proximal to the distal extremity thereof. In yet another alternatively preferred arrangement, and if desired, the substrate 34 optionally may suitably be mounted or carried on the exterior of catheter 22 substantially as indicated in FIG. 9. As in the prior embodiment of FIGS. 1-5, the substrate 34 of FIG. 7 may be hard wired to an analyzer or transmitter device by suitable conductors (not shown) extending along or through catheter 22. Alternatively, the transmitter device 46 may be employed or integrated directly on the substrate module 34 as will occur to those of ordinary skill in this art. As schematically indicated by FIG. 6, it will be appreciated that catheter 22 may be a peripheral IV catheter 22, or central venous catheters 22′ and 22″.

An important advantage of the present invention is that the administration of medications to patients may be automated by a feedback loop comprising the sensing microchip substrate 34, its associated communication device 46, an error signal detector (analyzer 42) and a known device for opening or closing the valve on an IV line in response to the generated error signal (not shown), thus causing the error signal to become nulled out and thereby permitting precise dosing of medicine on an individualized and substantially instantaneous basis. Similarly, the dosing of anesthesia drugs can be precisely and instantaneously monitored and adjusted to keep a patient at a level anesthetized state during surgery.

The present invention achieves many other benefits and advantages. Some of these include the following: (1) provides instantaneous blood (or other fluid) analysis at bed-side or on a surgical table obviating the need to send a sample to the lab and batching that sample with many others which may take hours or days; (2) permits customized patient treatment by drug dosing rather than relying upon standard protocols depending upon the patient's weight; (3) improves hospital efficiency by eliminating delays encountered in patient treatment due to a need to await test results; (4) allows for blood or bodily fluid testing at office-based surgery centers; and (5) makes feasible the combination of the present invention with standard I.C.U., E.R. and anesthesia monitors to permit patient testing throughout a hospital.

The “microchip substrate” contemplated by the present invention may be designated a “Biochip.”

The foregoing detailed description is considered as illustrative only of the principles of the invention. Numerous modifications and changes will readily occur to those skilled in the art and therefore, it is not desired to limit the invention to the exact construction and operation shown and described. For example, the microchip module may be cleaned (sterilized) and re-used, or may be disposed of after a single use. If desired, the syringe may be permanently attached to the module and provided as a single self-contained unit. Also, it will be appreciated that the invention may be employed in connection with conventional arterial lines or central venous lines. Accordingly, all suitable modifications and equivalents falling within the broad scope of the subject matter described above may be resorted to in carrying out the present invention.

What is claimed is:

1. An apparatus for measuring the physical and/or chemical characteristics of a bodily fluid comprising: a portable microchip substrate for sensing said physical and/or chemical characteristics of said bodily fluid upon contact therewith, a support for said microchip substrate capable of coming into contact with the bodily fluid of a patient, said support including at least in part a catheter insertable in a patient's body.

2. The apparatus of claim 1 wherein said substrate is mounted directly on said catheter.

3. The apparatus of claim 2 wherein said substrate is mounted directly on said catheter proximal to the distal extremity thereof.

4. The apparatus of claim 3 wherein said catheter has a lumen defining an interior wall surface and said microchip substrate is mounted on the wall surface of said lumen.

5. The apparatus of claim 3 wherein said microchip substrate is mounted on the exterior wall surface defined by said catheter.

6. The apparatus of claim 1 wherein said catheter is inserted in a vein on the patient's body and said bodily fluid is blood.

7. The apparatus of claim 1 wherein said support comprises at least in other part a receptacle, a needleless syringe, and a valveport on said receptacle, wherein said needleless syringe is adapted to be connected said valveport to draw said bodily fluid sample through said catheter and then through said receptacle into contact with said substrate.

8. The apparatus of claim 7 wherein said receptacle further includes a transmitter for transmitting sensed signals representative of said characteristics of said sample to a remote location.

9. The apparatus of claim 8 wherein said remote location defines an analyzer responsive to said sample.

10. The apparatus of claim 9 wherein said analyzer is adapted to generate an error signal, and said catheter is connected to a source of medication having an adjustable valve, and wherein said error signal is used to adjust said valve to apply medication to said patient until said error signal is nulled out.
